(19) United States
(12) Patent Application Publication
(10) Pub. No.: US 2008/0247957 A1
(43) Pub. Date: Oct. 9, 2008

(54) Advanced drug delivery strategy and platform for minimally-invasive treatment of liver cancer

(75) Inventor: Margaret A. Wheatley, Media, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Drexel University

(21) Appl. No.: 12/070,161

(22) Filed: Feb. 15, 2008

Related U.S. Application Data
(60) Provisional application No. 60/890,310, filed on Feb. 16, 2007.

(51) Int. Cl. A61K 49/00; A61K 9/50; A61P 43/00

(52) U.S. Cl. 424/9.5; 424/490

(57) ABSTRACT

The present invention comprises minimally invasive methods useful in the treatment of liver cancer. The method of the present invention comprises administering to an individual with liver cancer an injectable, echogenic nanocapsule and/or microcapsule wherein the nanocapsule and/or microcapsule comprises a therapeutic agent, a targeting moiety, or a combination thereof. The method of the present invention further comprises monitoring the distribution of the nanocapsule and/or microcapsule using ultrasound. The method of the invention delivers a therapeutic agent to a liver cancer cell either by biodegradation of a nanocapsule and/or microcapsule comprising a therapeutic agent, or by altering the biodegradation of a nanocapsule and/or microcapsule using ultrasound.

### CROSS REFERENCE TO RELATED APPLICATIONS

Application claims priority under 35 U.S.C. § 119(e) to U.S. Provisional Application No. 60/890,310, filed on Feb. 16, 2007, which application is incorporated by reference herein in its entirety.

### BACKGROUND OF THE INVENTION

Liver cancer is one of the most common and deadly malignancies in the world with upwards of 1.3 million new cases and 1 million deaths reported each year. Although liver cancer is far more prevalent in the developing world, its incidence is on the rise in the United States, Europe, and Japan. In addition to primary liver cancer, many other late-stage malignancies metastasize to the liver. Colorectal cancer is the most common source of live metastases with more than 118,000 new cases worldwide each year (American Lung Association Epidemiology and Statistic Unit Research and Program Services, May, 2005; Leonard et al., 2005, J. Clin. Oncol. 23:2038-2048).

Current treatments for liver cancers include systemic chemotherapy, cryo- or chemical-ablation, chemoembolization, and liver resection or transplant. These treatment modalities are highly specialized and require state-of-the-art medical facilities and highly skilled surgeons. Consequently, they are expensive in developed countries and largely unavailable in the developing world. In addition, these treatment modalities have several inherent clinical shortcomings including significant side effects and risk of damage to vulnerable tissue (especially heart) due to toxicity of therapeutic agents used in chemotherapy, as well as significant risk of infection and hemorrhage.

Clearly, new treatment modalities for liver cancer treatment that prolong life, improve patient quality of life, and provide better clinical outcomes are needed. The present invention meets this need.

### SUMMARY OF THE INVENTION

In one embodiment, the present invention comprises a method of selectively contacting a liver cancer cell with a therapeutic agent in an individual with liver cancer, the method comprising administering a composition comprising a polymer-based, echogenic nanocapsule and/or microcapsule to the individual, wherein the therapeutic agent is attached to, adsorbed to, encapsulated in, or any combination thereof, the nanocapsule and/or microcapsule, wherein the nanocapsule and/or microcapsule further comprises a targeting moiety, wherein the targeting moiety interacts with a target molecule present on a liver cancer cell, thereby contacting the liver cancer cell with the therapeutic agent. In one aspect, the therapeutic agent is released in the liver of the individual by degradation of said polymer-based nanocapsule and/or microcapsule. In another aspect, the individual is a human. In still another aspect, the degradation is enhanced by ultrasound. In still another aspect, the distribution of the nanocapsule and/or microcapsule comprising a therapeutic agent is monitored using ultrasound, wherein when the nanocapsule and/or microcapsule is detected in the liver, the selective release of the therapeutic agent in the liver is triggered by the rupture of the nanocapsule and/or microcapsule with ultrasound.

In another embodiment, the present invention also comprises a method of treating liver cancer, the method comprising the steps of: a) administering to an individual with liver cancer a composition comprising a nanocapsule and/or microcapsule, wherein the nanocapsule and/or microcapsule comprises a therapeutic agent, wherein the therapeutic agent either directly or indirectly inhibits the viability, growth, or proliferation of said cancer; b) monitoring the in vivo distribution of the nanocapsule and/or microcapsule using ultrasound to detect the nanocapsule and/or microcapsule in the liver; wherein when the nanocapsule and/or microcapsule is detected in the liver, the therapeutic agent is selectively released in the liver by enhancing the biodegradation of the nanocapsule and/or microcapsule by insonation of the liver. In one aspect, the individual is a human.

In yet another embodiment, the present invention also comprises a method of treating liver cancer, the method comprising the steps of: a) administering to an individual with liver cancer a composition comprising a nanocapsule and/or microcapsule, wherein the nanocapsule and/or microcapsule comprises a therapeutic agent, wherein the therapeutic agent either directly or indirectly inhibits the viability, growth, or proliferation of said cancer, and wherein the nanocapsule and/or microcapsule further comprises a targeting moiety, wherein the targeting moiety interacts with a target molecule present on a liver cancer cell; and b) monitoring the in vivo distribution of the nanocapsule and/or microcapsule using ultrasound to detect the nanocapsule and/or microcapsule is in the liver, wherein when the nanocapsule and/or microcapsule is detected in liver, the therapeutic agent is selectively released in the liver by enhancing the biodegradation of the nanocapsule and/or microcapsule by insonation of the liver: In one aspect, the individual is a human.

In still another embodiment, the present invention comprises method of treating liver cancer, the method comprising the steps of: a) administering to an individual with liver cancer a composition comprising at least one population of a nanocapsule and/or microcapsule; wherein the population essentially comprises a single therapeutic agent, a single targeting moiety, or a combination thereof, wherein the therapeutic agent either directly or indirectly inhibits the viability, growth, or proliferation of the cancer, and wherein the targeting moiety interacts with a target molecule present on a liver cancer cell; and b) monitoring the in vivo distribution of the population of a nanocapsule and/or microcapsule using ultrasound to detect the population is in the liver, wherein when the population is detected in liver, the therapeutic agent is selectively released in the liver by enhancing the biodegradation of the nanocapsule and/or microcapsule by insonation of the liver. In one aspect, the individual is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A and FIG. 1B, is a series of images depicting scanning electron microscopy (SEM) of the surface morphology of the capsules prepared with PVA of two different molecular weights (no camphor in preparation). FIG. 1A depicts capsules (DL25) prepared with the DL 3A polymer and 25,000 MW PVA (scale bar=1 μm). FIG. 1B depicts capsules (DL6) prepared with the DL 3A polymer and 6000 MW PVA (size bar=10 μm).

FIG. 2 is a graph depicting the cumulative dose response curves of capsules prepared with DL 3A polymer and 6000 MW PVA (DL6; ▪) and 25,000 MW PVA (DL25; ▴) n=3.

FIG. 3 is a graph depicting the cumulative dose response curves of capsules prepared with the DL 3A polymer, 25,000 MW PVA and different camphor amounts. DL25 0.0 g (▪), DL25C 0.025 g (♦), DL25C 0.05 g (▴), DL25C 0.1 g (X), and DL25C 2.5 g (□). n=3±deviation from the mean.

FIG. 4 is a graph depicting the noncumulative dose response curve of capsules (DL25AC) prepared with the DL3A polymer, 25,000 MW PVA, 0.05 g of camphor, and 4 wt/vol % ammonium carbonate aqueous solution. Capsule suspension insonated at (♦) 2.25, (▪) 5, (▴) 7.5, and (□) 10 MHz. n=3±deviation from the mean.

FIG. 5 is a graph depicting the effect of loading method upon amount of drug incorporated into capsules for PLA (gray bar) and PLGA (white bar) polymers. Drug is doxorubicin (DOX).

FIG. 6 is a graph depicting the effect of starting concentration of drug (wt % DOX) on amount incorporated into capsules depending on polymer in use; PLA (gray bar) and PLGA (white bar).

FIG. 7 is an image depicting SEM of microcapsules and/or nanocapsules loaded with sudan black, a model for the chemotherapeutic agent, Paclitaxel. Bar=2 μm.

FIG. 8, comprising FIG. 8A and FIG. 8B, is a series of graphs depicting the results of a new method (pre-hardening adsorption) of drug loading and compares them to previous methods of drug loading. FIG. 8A is a graph depicting a comparison of loading efficiency for pre-hardening adsorption with previous methods of drug loading using different starting concentrations of DOX. FIG. 8B is a graph depicting a comparison of echogenicity of nanocapsules and/or microcapsules that had been loaded with drug payloads using different methods.

FIG. 9, comprising FIG. 9A and FIG. 9B, is a series of graphs depicting the acoustic characterization of drug-loaded microcapsules and/or nanocapsules. FIG. 9A is a graph depicting the dose response relationship between enhancement (dB) and dose. FIG. 9B is a graph depicting the relative stability of the echogenic properties of nanocapsules and/or microcapsules loaded with drug.

FIG. 10, comprising FIG. 10A and FIG. 10 B, is a series of graphs comparing the acoustic properties for drug loaded at different temperatures. FIG. 10 A is a graph depicting the dose-response curve for 3% DOX loaded by cold adsorption (4° C.) at 24 hours. FIG. 10B is a graph comparing the acoustic properties of 3% DOX loaded at room temperature and measured at various times.

FIG. 11, comprising FIG. 11A and FIG. 11B is an ultrasound image of rabbit kidney. FIG. 11A is an image taken before injection of the nanocapsules and/or microcapsules. The kidney is poorly visible. FIG. 11B is an ultrasound image taken after injection of the nanocapsules and/or microcapsules. The internal structure of is greatly enhanced.

FIG. 12 comprising FIG. 12A and FIG. 12 B is a series of images depicting Micro Flow Doppler imaging of a subcutaneous melanoma tumor (boxed area).

FIG. 12A is an image taken before injection of DOX-loaded nanocapsules and/or microcapsules. FIG. 12B is an image taken of the same region after injection of 0.1 ml/kg of DOX-loaded nanocapsules and/or microcapsules. Note that the loaded nanocapsules and/or microcapsules penetrate the tumor. Also nanocapsules and/or microcapsules are observed vessel feeding the tumor (lower right of box area).

FIG. 13 is a graph depicting ultrasound-triggered drug release from drug-loaded nanocapsules and/or microcapsules as a function of time (minutes). Different frequencies were tested as follows: ▴ stirred control, ♦ 5 MHz, ▪ 7.5 MHz, 10 MHz.

### DETAILED DESCRIPTION OF THE INVENTION

The present invention is based, in part, upon the discovery of a minimally-invasive method of delivering therapeutic agents directly to liver cancer cells. The present invention comprises an injectable, echogenic microcapsule and/or nanocapsule as disclosed in U.S. Pat. No. 5,352,436, U.S. Pat. No. 5,955,143, and U.S. Patent Application 2004/0161384, all of which are incorporated by reference herein in their entirety.

In one embodiment, the present invention includes a microcapsule and/or nanocapsule wherein the microcapsule and/or nanocapsule comprise a therapeutic agent. In another embodiment, the present invention includes a microcapsule and/or nanocapsule wherein the microcapsule and/or nanocapsule comprise a targeting moiety that directs the capsule specifically to a liver cancer cell. In still another embodiment, the present invention includes a microcapsule and/or nanocapsule wherein the microcapsule and/or nanocapsule comprise a therapeutic agent and a targeting moiety, wherein the targeting moiety directs the capsule specifically to a liver cancer cell.

The present invention further comprises methods for the treatment of liver cancer. In one embodiment, the present invention comprises a method of delivering a therapeutic agent to a liver cancer cell wherein a microcapsule and/or a nanocapsule comprising a therapeutic agent is administered to the individual with liver cancer. Once administered to the individual, a microcapsule and/or nanocapsule of the invention undergoes a process of biodegradation, thereby releasing the therapeutic agent. Another embodiment of the present invention comprises a method of selectively releasing a therapeutic agent directly in the liver using ultrasound to trigger the localized rupture of the nanocapsule and/or microcapsules. Still another embodiment of the present invention comprises a method of targeting delivering a therapeutic agent directly to a liver cancer cell wherein a microcapsule and/or a nanocapsule comprising a therapeutic agent and a targeting moiety is administered to an individual with liver cancer wherein the targeting moiety specifically binds to a target molecule present on a liver cancer cell, and wherein the microcapsule and/or a nanocapsule undergoes a process of biodegradation in the liver. In yet another embodiment, a method of the invention comprises administering a microcapsule and/or nanocapsule comprising a therapeutic agent and a targeting moiety, to an individual with liver cancer, wherein the targeting agent binds specifically to a target molecule present on a liver cancer cell, and wherein the therapeutic agent is selectively released in the liver using ultrasound.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The term “cancer” is used throughout the specification to refer to cells that have undergone a malignant transformation that makes them pathological to the host organism. Primary cancer cells (that is, cells obtained from or near the site of malignant transformation) can be readily distinguished from non-cancerous cells using well-established techniques, particularly histological examination. The pathological transformation into a cancer cell results in the formation and growth of a cancerous or malignant neoplasm, i.e., abnormal tissue that grows by cellular proliferation, often more rapidly than normal and continues to grow after the stimuli that initiated the new growth cease. Malignant neoplasms exhibit partial or complete lack of structural organization and functional coordination with the normal tissue and most invade surrounding tissues, metastasize to several sites, and are likely to recur after attempted removal and to cause the death of the patient unless adequately treated.

“Liver cancer,” as used herein refers to any primary cancer of the liver, including but not limited to hepatocellular carcinoma, or secondary cancer of the liver, including metastatic carcinoma of any origin in the liver.

As used herein, “echogenic” means that the microcapsule or nanocapsule is capable of producing a detectable echo when insonated with ultrasonic waves due to an acoustic impedance mismatch between blood and the microcapsule or nanocapsule. In a preferred embodiment, echogenic characteristics result from the microcapsule and/or nanocapsule being hollow and/or porous. By “porous” for purposes of the present invention, it is meant that the capsules contain one or more pores.

As used herein, to “treat” means reducing the frequency with which symptoms of a disease, defect, disorder, or adverse condition, and the like, are experienced by a patient. In the present invention, the disease being treated is liver cancer.

As used herein, a “therapeutically effective amount” is the amount of a composition of the invention sufficient to provide a beneficial effect to the individual to whom the composition is administered. For instance, with regard to the administration of microcapsules and/or nanocapsules comprising a therapeutic agent to an individual, a “therapeutically effective amount” is the amount of microcapsules or nanocapsules which is sufficient to provide a beneficial effect to the subject to which the microcapsules and/or nanocapsules are administered.

A “therapeutic” treatment is a treatment administered to a subject who exhibits at least one symptom of liver cancer for the purpose of treating or alleviating the at least one symptom.

A “therapeutic agent” of the present invention is any agent useful in treating liver cancer. Therapeutic agents of the present invention include agents that directly affect the viability, growth, or proliferation of a liver cancer cell, including but not limited to chemotherapeutic compounds. Therapeutic agents of the present invention also include agents that indirectly affect the viability, growth, or proliferation of a liver cancer cell, such as any compound or molecule that renders a liver cancer cell more susceptible to a chemotherapy or radiation therapy.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well.

A “composition,” as used herein, is intended to mean a microcapsule or a nanocapsules comprising a therapeutic agent and optionally another compound or composition, inert (for example, a detectable agent or label) or active, such as an adjuvant.

A “pharmaceutical composition” is intended to include a microcapsule and/or a nanocapsule that comprises an therapeutic agent with a carrier, inert or active, making the composition suitable for diagnostic or therapeutic use in vitro, in vivo or ex vivo.

By “pharmaceutically acceptable carrier” is meant any carrier, diluent or excipient which is compatible with the biological component of a pharmaceutical composition and not deleterious to the recipient.

By “population of a nanocapsule and/or microcapsule,” as used herein, it is meant an essentially homogeneous group of nanocapsules and/or microcapsules prepared so that they comprise a single targeting moiety, a single therapeutic agent, or a combination thereof.

### DESCRIPTION

The present invention comprises compositions and methods useful in the treatment of an individual with liver cancer. In one embodiment of the invention, the individual with liver cancer is a mammal preferably a human.

The present invention comprises an injectable, echogenic nanocapsule and/or microcapsule that can be administered to an individual diagnosed with liver cancer. The echogenic properties of a nanocapsule and/or microcapsule of the present invention make it detectable in vivo using standard clinical ultrasound. A nanocapsule and/or microcapsule of the present invention may comprise a therapeutic agent useful in the treatment of liver cancer. A nanocapsule and/or microcapsule may further comprise a targeting moiety which directly interacts with a target molecule present in or on a liver cancer cell.

The methods of the present invention comprise targeting delivery of a therapeutic agent to a liver cancer cell to minimize deleterious side effects associated with many therapeutic agents used in the treatment of liver cancer. Accordingly, delivery of a therapeutic agent may be targeted to a liver cancer cell by incorporating a targeting moiety that binds to a target molecule on a liver cancer cell onto a nanocapsule and/or microcapsule of the present invention. A therapeutic agent may also be selectively released in the liver by insonation of the liver or a cancer therein to enhance the biodegradation of a nanocapsule and/or microcapsule comprising a therapeutic agent.

### I. Compositions

A. Microcapsules and Nanocapsules

A composition of the present invention comprises an injectable, echogenic nanocapsule and/or microcapsule, for example disclosed in U.S. Pat. No. 5,352,436, U.S. Pat. No. 5,955,143, and U.S. Patent Application 2004/0161384, all of which are incorporated by reference herein in their entirety. The nanocapsules and/or microcapsules of the present invention comprise a biocompatible, biodegradable polymer including polyhydroxy acid polymers such as poly-lactic-co-glycolic acid and poly-L-Lactic acid.

The microcapsules and nanocapsules of the present invention can be modified to be loaded with a therapeutic agent useful in the treatment of liver cancer. Further, the microcapsules and nanocapsules of the present invention can be modified on their surface with a targeting moiety that specifically targets the microcapsule and/or nanocapsule to selected tissue types. These microcapsules and nanocapsules of the present invention are capable of extravasation to specific tissues, such as a tumor in the liver, and are capable of functioning as an ultrasound contrast agent. The nanocapsules and microcapsules of the present invention can also be used to carry and deliver a therapeutic agent to a specific target in the body, such as a liver cancer cell. Furthermore, these nanocapsules and microcapsules can be used to release a therapeutic agent at a selected target through an ultrasound triggering mechanism and/or rate predetermined biodegradation.

For purposes of the present invention, by “nanocapsule” it is meant a capsule sufficiently small in size to access the microvasculature of the human body. Nanocapsules of the present invention range in size from about 10 nm to about 500 nm, while microcapsules of the present invention range in size from about 500 nm to about 1000 microns. Nanocapsules of this size provide an advantage in that they can access areas difficult if not impossible to reach with microcapsules. For example, nanocapsules can pass through leaky tumor vasculature. In addition, nanocapsules have different resonance frequencies thus providing advantages in both imaging and delivery of therapeutic agents.

B. Therapeutic Agents

In one embodiment, a therapeutic agent is incorporated into the polymer-based nanocapsules or microcapsules of the present invention. Therapeutic agents may be adsorbed to and/or attached to the surface of the nanocapsule and/or microcapsule. To adsorb a drug product to the nanocapsule or microcapsule surfaces, the drug is dissolved in distilled water or a buffer, and then the dried nanocapsules or microcapsules are suspended in distilled water with the drug. The suspension is stirred overnight and then centrifuged to collect capsules. The resulting nanocapsules or microcapsules are then washed, frozen and lyophilized. The lyophilized nanocapsules or microcapsules have the drug product to be delivered adsorbed to their surfaces.

Therapeutic agents can also be attached to the nanocapsules or microcapsules in accordance with well known methods for conjugation. Alternatively, or in addition, a therapeutic agent can be encapsulated in the nanocapsules or microcapsules. Water soluble therapeutic agents can be encapsulated in the nanocapsules or microcapsules by including water during emulsification and dissolving the bioactive agent in this water forming a w/o/w emulsion system. Further, a water soluble, lyophilizable agent such as ammonium carbonate or ammonium carbamate can be included in the water phase, to increase echogenicity of the agents. This is removed during freeze drying. Non-water soluble therapeutic agents can be encapsulated in the nanocapsules by dissolving the therapeutic compound in the non-polar organic solvent in the first step of preparation of these capsules.

Examples of therapeutic agents which can be adsorbed, attached and/or encapsulated in a microcapsules and/or nanocapsules of the present invention include, but are not limited to, antineoplastic and anticancer agents useful in the treatment of liver cancer. Non-limiting examples of such drugs include doxorubicin, cisplatin, methotrexate, 5FU (fluorouracil), irinotecan (topoisomerase 1 inhibitor), 3-bromo-pyruvate. The present invention should not be construed to be limited to these agents, but rather to include any therapeutic agent, both known and unknown, useful in treating liver cancer. Further, a therapeutic agent useful in the present invention includes any drug or molecule that renders a cancer cell susceptible to radiation or chemotherapy.

In one embodiment of the present invention, a population of nanocapsules and/or microcapsules is prepared wherein a single therapeutic agent is adsorbed, attached and/or encapsulated in a microcapsule and/or microcapsule, thus formulating an essentially homogeneous population of nanocapsules or microcapsules comprising a single therapeutic agent. In another embodiment of the present invention, an essentially homogenous population of nanocapsules and/or microcapsules is prepared wherein the population comprises a single targeting moiety. In yet another embodiment of the present invention, an essentially homogenous population of nanocapsules and/or microcapsules is prepared wherein the population comprises a single combination of a therapeutic agent and a targeting moiety. Accordingly, a patient with liver cancer may be administered a population of nanocapsules and/or microcapsules wherein the population essentially comprising a single therapeutic agent, a single targeting moiety, or a single combination thereof.

In another embodiment, it will be appreciated by a skilled artisan that multiple populations of nanocapsules and/or microcapsules may be prepared according to the methods of the present invention wherein each population comprises a different therapeutic agent, a different targeting moiety, or a different combination thereof. Thus, a patient with liver cancer may be administered multiple populations of nanocapsules and/or microcapsules, each comprising a different therapeutic agent, a different targeting moiety, or a different combination thereof. These different populations of nanocapsules and/or microcapsules may be administered simultaneously or at different times during the course of an individual's treatment.

The nanocapsules and/or microcapsules of the present invention can be used in combination with other treatment regimens, including, but not limited to, virostatic and virotoxic agents, antibiotic agents, antifungal agents, anti-inflammatory agents (steroidal and non-steroidal), antidepressants, anxiolytics, pain management agents, (acetaminophen, aspirin, ibuprofen, opiates (including morphine, hydrocodone, codeine, fentanyl, methadone), steroids (including prednisone and dexamethasone), and antidepressants (including gabapentin, amitriptyline, imipramine, doxepin) antihistamines, antitussives, muscle relaxants, brondhodilaters, beta-agonists, anticholinergics, corticosteroids, mast cell stabilizers, leukotriene modifiers, methylxanthines, nucleic acid based therapeutic agents including gene therapy vectors and other genetic materials, and peptide inhibitors, which when localized to tumors prevent tumor growth, as well as combination therapies, and the like.

The compositions of the present invention may be administered before, during, after, or throughout administration of any other therapeutic agents used in the treatment of the individual patient's liver cancer.

C. Target Molecules

A “target molecule” of the present invention is any gene, protein, or metabolite present in or on a liver cancer cell that interacts with a targeting moiety to direct a nanocapsule and/or microcapsule of the present invention to a liver cancer cell according to the method of the invention. Such target molecules include DNA comprising the entire or partial sequence of the nucleic acid sequence encoding the target molecule, or the complement of such a sequence. Nucleic acids useful as target molecules in the invention should be considered to include both DNA and RNA comprising the entire or partial sequence of any of the nucleic acid sequences of interest. A target molecule protein should be considered to comprise the entire or partial amino acid sequence of any of the target molecule proteins or polypeptides.

It is understood by those of skill in the art that a target molecule used to direct a nanocapsule and/or microcapsule of the present invention to a liver cancer cell generally refers to a protein or a nucleic acid encoding a protein expressed by a liver cancer cell that can be detected using the method of the present invention.

Some non-limiting examples of target molecules upregulated in liver cancer include diubiquitin; PTTG1, pituitary tumor-transforming gene 1, or securin; Galectin 3, LGALS3, one of a family of beta-galactoside-binding animal lectins; solute carrier family 2, member 3, or glucose transporter 3 (GLUT3); claudin 4, also known as clostridium perfringens enterotoxin receptor 1; occluding; Serine protease inhibitor, Kazal type I (SPINKI), also called pancreatic secretory trypsin inhibitor (PSTI) or tumor-associated trypsin inhibitor (TATI); Midkine; Stathmin, leukemia-associated phosphoprotein 18. A skilled artisan will appreciate that any protein or nucleic acid, both known or unknown, that can be used to distinguish a liver cancer cell from a normal liver cell, and interact with a targeting moiety present on a nanocapsule and/or microcapsule would be useful as a target molecule in the present invention.

D. Targeting Moieties

A target molecule is characterized by its interaction with a targeting moiety. A “targeting moiety,” as used herein, may be an antibody, a naturally-occurring ligand for a receptor or functional derivatives thereof, a vitamin, a small molecule mimetic of a naturally-occurring ligand, a peptidomimetic, a polypeptide or aptamer, or any other molecule provided it binds specifically to a target molecule, or a fragment thereof.

A targeting moiety may, either directly or indirectly, comprise a detectable tag or label, thereby labeling a cell comprising a target molecule of interest. Labeling may be accomplished by any method known to those of skill in the art. For example, an antibody directed to a target molecule may comprise a detectable tag or label. In another example, an antibody directed to a target molecule may be contacted by a second antibody comprising a detectable tag or label. The label may be fluorochromated anti-target molecule antibody, which may include but is not limited to a magnetic bead-, colloidal bead-, FITC-, AMCA-, fluorescent particle-, or liposome-conjugated antibodies.

1. Targeting Moiety—Antibodies

When the antibody used as a targeting moiety in the compositions and methods of the invention is a polyclonal antibody (IgG), the antibody is generated by inoculating a suitable animal with the targeted cell surface molecule. Antibodies produced in the inoculated animal which specifically bind to the cell surface molecule are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow, et al. (1998, In: Antibodies, A Laboratory Manual, Cold Spring Harbor, N.Y.).

Monoclonal antibodies directed against a full length targeted cell surface molecule or fragments thereof may be prepared using any well known monoclonal antibody preparation procedures, such as those described, for example, in Harlow et al. (1998, In: Antibodies, A Laboratory Manual, Cold Spring Harbor, N.Y.) and in Tuszynski et al. (1988, Blood, 72:109-115). Human monoclonal antibodies may be prepared by the method described in U.S. Patent Publication 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein.

When the antibody used in the methods of the invention is a biologically active antibody fragment or a synthetic antibody corresponding to antibody to a targeted cell surface molecule, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3,4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with targeted cell surface molecule epitopes. These antibodies are capable of binding to the targeted cell surface molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen, et al. (U.S. Pat. No. 6,180,370), Wright et al., (supra) and in the references cited therein, or in Gu et al. (1997, Thrombosis and Hematocyst 77(4):755-759), or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to the targeted cell surface molecule. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1998, Using Antibodies: A Laboratory Manual, Cold Spring Harbor Laboratory Press, NY).

V_{H} proteins isolated from other sources, such as animals with heavy chain disease (Seligmann et al., 1979, Immunological Rev. 48:145-167, incorporated herein by reference in its entirety), are also useful in the compositions and methods of the invention. The present invention further comprises variable heavy chain immunoglobulins produced from mice and other mammals, as detailed in Ward et al. (1989, Nature 341:544-546, incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful as targeting moieties in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

One of skill in the art will appreciate that it may be desirable to detect more than one protein of interest in a biological sample. Therefore, in particular embodiments, at least two antibodies directed to two distinct proteins are used. Where more than one antibody is used, these antibodies may be added to a single sample sequentially as individual antibody reagents or simultaneously as an antibody cocktail. Alternatively, each individual antibody may be added to a separate sample from the same source, and the resulting data pooled.

2. Targeting Moieties-Protein, Peptide, and Polypeptide

Other types of targeting moieties useful in the invention comprise a protein, peptide or polypeptide and may be obtained using standard methods known to the skilled artisan. Such methods include chemical organic synthesis or biological means. Biological means include purification from a biological source, recombinant synthesis and in vitro translation systems, using methods well known in the art.

A peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the α-amino protecting group, and the FMOC method, which utilizes 9-fluorenylmethyloxcarbonyl to protect the α-amino of the amino acid residues. Both methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use as a targeting moiety, a peptide may be purified to remove contaminants. In this regard, it will be appreciated that the peptide can be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Antibodies and other peptide targeting moieties may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

3. Targeting Moieties—Nucleic Acids

Any number of procedures may be used for the generation of targeting moieties comprising nucleic acids using recombinant DNA methodology well known in the art (see Sambrook et al., 2001, Molecular Cloning: A Laboratory Manual, Cold Spring Harbor Laboratory Press, New York; Ausubel et al., 2001, Current Protocols in Molecular Biology, Green & Wiley, New York) and by direct synthesis. For recombinant and in vitro transcription, DNA encoding RNA molecules can be obtained from known clones, by synthesizing a DNA molecule encoding an RNA molecule, or by cloning the gene encoding the RNA molecule. Techniques for in vitro transcription of RNA molecules and methods for cloning genes encoding known RNA molecules are described by, for example, Sambrook et al. (2001, Molecular Cloning: A Laboratory Manual, Cold Spring Harbor Laboratory Press, Cold Spring Harbor, N.Y.).

An isolated nucleic acid targeting moiety of the present invention can be produced using conventional nucleic acid synthesis or by recombinant nucleic acid methods known in the art and described elsewhere herein (Sambrook et al., 2001, Molecular Cloning: A Laboratory Manual, Cold Spring Harbor Laboratory Press, Cold Spring Harbor, N.Y.) and Ausubel et al. (2001, Current Protocols in Molecular Biology, Green & Wiley, New York).

As an example, a method for synthesizing nucleic acids de novo involves the organic synthesis of a nucleic acid from nucleoside derivatives. This synthesis may be performed in solution or on a solid support. One type of organic synthesis is the phosphotriester method, which has been used to prepare gene fragments or short genes. In the phosphotriester method, oligonucleotides are prepared which can then be joined together to form longer nucleic acids. For a description of this method, see Narang, et al., (1979, Meth. Enzymol., 68: 90) and U.S. Pat. No. 4,356,270. The phosphotriester method can be used in the present invention to synthesize an isolated snRNA.

In addition, a nucleic acid targeting moiety of the present invention can be synthesized in whole or in part, or an isolated snRNA can be conjugated to another nucleic acid using organic synthesis such as the phosphodiester method, which has been used to prepare a tRNA gene. See Brown, et al. (1979, Meth. Enzymol., 68: 109) for a description of this method. As in the phosphotriester method, the phosphodiester method involves synthesis of oligonucleotides which are subsequently joined together to form the desired nucleic acid.

A third method for synthesizing a nucleic acid targeting moiety (U.S. Pat. No. 4,293,652) is a hybrid of the above-described organic synthesis and molecular cloning methods. In this process, the appropriate number of oligonucleotides to make up the desired nucleic acid sequence is organically synthesized and inserted sequentially into a vector which is amplified by growth prior to each succeeding insertion.

In addition, molecular biological methods, such as using a nucleic acid as a template for a PCR or LCR reaction, or cloning a nucleic acid into a vector and transforming a cell with the vector can be used to make large amounts of the nucleic acid of the present invention.

E. Pharmaceutical Compositions

Compositions comprising a nanocapsule and/or microcapsule disclosed herein, may be formulated and administered to a mammal diagnosed with liver cancer for the purpose of directly inhibiting the viability, growth, or proliferation of a liver cancer cell. In addition, compositions comprising a nanocapsule and/or microcapsule disclosed herein, may be formulated and administered to a mammal diagnosed with liver cancer for the purpose of indirectly inhibiting the viability, growth, or proliferation of a liver cancer cell. A non-limiting example of indirectly inhibiting the viability, growth, or proliferation of a liver cancer cell includes rendering a cancer cell susceptible to chemotherapeutic and radiation therapy.

The invention encompasses the preparation and use of pharmaceutical compositions comprising a nanocapsules or microcapsule as described herein wherein the nanocapsules or microcapsule further comprises a compound useful for treatment of liver cancer as an active ingredient. Such a pharmaceutical composition may consist of a nanocapsule or microcapsule alone, in a form suitable for administration to a subject, or the pharmaceutical composition may comprise a nanocapsules or microcapsule and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing a nanocapsule or microcapsule comprising an active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the invention may be prepared, packaged, or sold in formulations suitable for oral, parenteral, buccal, or another route of administration.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of a non-limiting example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intravenous, intra-arterial, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### II. Methods

The present invention comprises minimally invasive methods of treating liver cancer. In one embodiment, the present invention comprises selectively releasing a therapeutic agent in the liver wherein the therapeutic agent is attached to, adsorbed to, encapsulated in, or any combination thereof, a nanocapsule and/or microcapsule. In another embodiment, the present invention comprises targeting a nanocapsule and/or microcapsule comprising a therapeutic agent and a targeting moiety to a liver cancer cell.

Accordingly, the methods of the present invention comprise administering an echogenic nanocapsule and/or microcapsule to an individual diagnosed with liver cancer. Once administered to an individual, a microcapsule and/or nanocapsule comprising a therapeutic agent, a targeting moiety, or a combination thereof undergoes a process of biodegradation, thereby releasing the therapeutic agent in vivo. In one embodiment, the invention comprises a method of selectively releasing a therapeutic agent in the liver. The method comprises detecting a nanocapsule and/or microcapsule in the liver using standard, clinical ultrasound, then selectively releasing the therapeutic agent in the liver by enhancing the degradation of the nanocapsule and/or microcapsule by insonating the liver or cancer therein.

In another embodiment, the methods of the present invention comprise targeting delivery of a therapeutic agent to a liver cancer cell by administering a nanocapsule and/or microcapsule comprising therapeutic agent and a targeting moiety that binds to a liver cancer cell. Once bound to a liver cancer cell, the nanocapsules and/or microcapsules comprising a targeting moiety will undergo biodegradation, thereby releasing the therapeutic agent in the liver. Alternatively, once bound to a liver cancer cell, the biodegradation of a nanocapsule and/or microcapsule comprising a targeting moiety can be enhanced using ultrasound to trigger the rupture of the nanocapsule and/or microcapsule in the liver. It will be appreciated by a skilled artisan that a method of treating liver cancer comprising more localized delivery of a therapeutic agent to the liver would minimize the deleterious side effects of many therapeutic agents.

In one embodiment, a method of the present invention comprises administering a single population of nanocapsules and/or microcapsules to an individual with liver cancer, wherein the population comprises a single therapeutic agent, a single targeting moiety, or a combination thereof. In another embodiment, a method of the present invention comprises administering multiple populations of nanocapsules and/or microcapsules to an individual with liver cancer.

In one embodiment of the invention, a therapeutic agent is administered to an individual with liver cancer wherein the therapeutic agent is adsorbed, attached and/or encapsulated in a microcapsule and/or nanocapsule of the present invention. In another embodiment of the invention, a free therapeutic agent is administered to an individual with liver cancer in conjunction with a nanocapsule and/or a microcapsule of the present invention, wherein the nanocapsule and/or a microcapsule comprises a therapeutic agent useful in the treatment of liver cancer.

A “free therapeutic agent,” as used herein, is a therapeutic agent not adsorbed, attached and/or encapsulated in a nanocapsule or microcapsule of the present invention. The free therapeutic agent may be administered before, during, or after the administration of a nanocapsule and/or a microcapsule of the present invention.

In still another embodiment of the present invention, a free therapeutic agent may be administered to an individual with liver cancer in a composition further comprising a nanocapsule and/or microcapsule of the present invention where the nanocapsule and/or microcapsule comprises a therapeutic agent, a targeting moiety, or any combination thereof. When a nanocapsule and/or microcapsule of the present invention comprising a therapeutic agent and is administered to an individual with liver cancer in conjunction with a free therapeutic agent, it will be appreciated by a skilled artisan that the therapeutic agent adsorbed, attached and/or encapsulated in a microcapsule and/or nanocapsule of the present invention need not be the same as the free therapeutic agent being administered. By way of a non limiting example, an individual with liver cancer may be administered free cisplatin in combination with a population of nanocapsules and/or a microcapsules wherein the nanocapsules and/or a microcapsules are conjugated to doxorubicin.

A. Method of Making a Microcapsule and/or Nanocapsule of the Invention

The polymer-based nanocapsules or microcapsules of the present invention can be prepared in accordance with the following method. A biocompatible, biodegradable polymer is dissolved in a solution comprising an oil phase and a substance soluble in the oil phase and easy to sublime in the lyophilizer. If the oil phase is an organic solvent such as acetone, this sublimable substance may be camphor, ammonium carbamate, theobromide, camphene or napthalene. An emulsion of large beads or capsules of mixed polymer and a sublimable substance such as camphor is then formed in the solution by probe sonication. The resulting emulsion is poured into a surfactant solution, preferably a 1% solution of polyvinyl alcohol, and homogenized to remove the oil phase, for example acetone from the capsules, causing them to shrink in size. The addition of the surfactant allows the breakup of the polymer/sublimable substance beads or capsules into smaller ones, thus enhancing the size reduction of the capsules. The emulsion is then washed with deionized water to remove additional acetone and dry the capsules. The capsules are then collected by centrifugation, washed, and re-collected by centrifugation. The washed capsules are then frozen at −85° C. for approximately 30 minutes and dried, preferably by lyophilization to remove any additional sublimable substance.

Alternatively, microcapsules and/or nanocapsules of the present invention can be prepared by a double emulsion or w/o/w emulsion process. In the process, the sublimable substance such as camphor is dissolved with a biocompatible, biodegradable polymer such as PLA in an oil phase such as acetone. A first emulsion is then generated by addition of ammonium carbonate followed by sonication. This first emulsion (w/o) is then poured into a surfactant solution such as PVA and homogenized. The double emulsion (w/o)/w in then poured into water and stirred. Resulting capsules and collected via centrifugation, washed, and lyophilized variation in parameters such as sonication time, homogenization time and polymer blend as well as concentrations of ammonium carbonate alters the capsule size.

B. Therapies

The nanocapsules and microcapsules of the present invention can be used in combination with other treatment modalities, such as chemotherapy, cryotherapy, hyperthermia, radiation therapy, surgical resection, and the like. Chemotherapy and radiation are commonly used as components of a combined modality treatment, and the choice of chemotherapeutic agent(s) and type and course of radiation therapy is generally governed by the characteristics of the individual cancer and the response of the individual. They can also be combined with both methods of treatment in the same course of therapy. Accordingly, the present invention encompasses combinations of the methods discussed above.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Materials

Poly-lactic-co-glycolic acid (PLGA) (Medisorb 5050 □L 3A, llot 9138-408) is commercially available (Alkermes. Poly (D,L-lactide-co-glycolic acid 50:50, PLGA) (Resomer RG 504H, lot 34020) was purchased from Boehringer Ingelheim. Poly(vinyl alcohol) (PVA), 80% mole hydrolyzed with a Mw of 6000, 88% mole hydrolyzed with a Mw of 25,000, and 99% mole hydrolyzed with a Mw of 133,000 were from Polysciences, Inc. (1R)-(+)-Camphor was from Sigma (St. Louis, Mo.) and ammonium carbonate powder was from J. T. Baker. All other chemicals were reagent grade from Fisher.

### Methods

### Capsule Fabrication

A. Double Emulsion Method for Microcapsule and/or Nanocapsule Fabrication

Microcapsules were prepared by an adapted double emulsion (W/O)/W solvent evaporation process. 28 PLGA (0.5 g) was dissolved in 20 mL of methylene chloride. To generate the first W/O emulsion, 1.0 mL of deionized water was added to the polymer solution and probe sonicated (using a XL-series, Misonix Incorporated sonicator) at 110 W for 30 s. The W/O emulsion was then poured into a 5% PVA (MW varied) solution and homogenized (using PT-3100 Homogenizer, Brinkmann Instruments, with a PTDA3020/2 sawtooth blade) for 5 min at 9500 rpm. The PVA acts as a surfactant and reduces the surface tension, whereas simultaneous homogenization breaks the W/O emulsion into a population of small beads. The double (W/O)/W emulsion was then poured into a 2% isopropanol solution and stirred at room temperature for 1 h to evaporate off the methylene chloride and thus harden the capsules. The capsules were collected by centrifugation, washed one time with deionized water, centrifuged (at 15° C. for 5 min at 5000 g) and the supernatant discarded. The capsules were then washed three times with hexane to further extract the methylene chloride from the polymer beads. The capsules can then be frozen in a −85° C. freezer and lyophilized using a Virtis Benchtop freeze dryer to fully dry the capsules and sublime the encapsulated water.

B. Double Emulsion Method for Microcapsule and/or Nanocapsule Fabrication: Preparation with Camphor

Camphor is a nonwater-soluble substance and is encapsulated in the oil phase. Different amounts of camphor were weighed and dissolved in the methylene chloride before the addition of the polymer. The capsule fabrication protocol is then followed as stated above. Capsules of mixed camphor and polymer are formed prior to lyophilization. Camphor and water sublime when freeze dried, leaving a void in their place, thus forming a hollow nanocapsule and/or microcapsule.

C. Double Emulsion Method for Microcapsule and/or Nanocapsule Fabrication: Preparation with Camphor and Ammonium Carbonate

Ammonium carbonate is a water-soluble substance and is encapsulated in the water phase. To generate the first w/o emulsion, deionized water was replaced with a 4 w/v % ammonium carbonate solution, which was added to the polymer/camphor methylene chloride solution and probe sonicated. The rest of the method of producing the capsules was not altered; however, the temperature of PVA was investigated. Capsules were prepared with three different PVA temperatures to determine the effects on capsule echogenicity. Capsules of mixed polymer, camphor, and ammonium carbonate were produced before freeze drying. Camphor, water, and ammonium carbonate sublime when freeze dried, leaving a void in their place and producing hollow PLGA microcapsules.

### Determination of Size Distribution

Static light scattering using a Horiba LA-910 Particle Size Analyzer (Horiba Instrument) was used to measure the size distribution of the microcapsules. Deionized water was used in the Horiba sample cell as a blank before each reading. The microcapsules were added in the sample cell (sufficient amount indicated by the Horiba LA-910 software). The settings, agitation speed of 4 and circulation speed of 3, were set on the machine using the windows based software interface.

### Cross Sectioning and Scanning Electron Microscopy (SEM)

Dried capsules were suspended in deionized water, subjected to probe sonication (110 W) for 5 min and freeze dried. SEM (Amray model 1830 D, Amray, Bedford, Mass.) was used to examine the surface morphology and cross sections of the capsules. The dried microcapsules were mounted on metal stubs with double-sided electrical tape. They were gold coated under reduced pressure with a sputter coater and viewed under the SEM at 20 kV.

In Vitro Testing of Acoustic Properties of Nanocapsules and/or Microcapsules

A one-dimensional pulsed A-mode ultrasound set-up was used with single-element, (broadband, 12.7 mm, element diameter, 50.8 mm) spherically focused transducers with center frequencies of 2.25, 5, 7.5, and 10 MHz (Panametrics, Inc., Waltham, Mass.). The −6 dB bandwidth of the transducers were 89%, 92%, 71%, and 65%, respectively. The transducers were inserted in a deionized water bath (25° C.) and focused through an acoustic window of a 100-mL custom-made sample vessel. A pulser/receiver (model 5072 PR, Panametrics, Inc., Waltham, Mass.) was used to pulse the transducers at a pulse repetition frequency of 100 Hz. The received signals were amplified to 40 dB and fed to the digital oscilloscope (Lecroy 9350A, Lecroy, Chestnut Ridge, N.Y.). The digitized data were stored and analyzed using Labview (National Instruments, Austin, Tex.).

The sample vessel was made from two 50-mL plastic centrifuge tubes (diameter=30 mm, length=115 mm, VWR Scientific, West Chester, Pa.), one with the end cut off and attached one on top of the other (cut end to open end) to allow a total volume of 100 mL, and the ability to securely close with a screw cap. The water tank was equipped with a stable mounting for the sample vessel. The microcapsules were weighed, suspended in phosphate-buffered saline solution (PBS) pH 7.4 (25° C.) and stirred with a magnetic stirrer. The reference (PBS) was taken as an average of six values.

### Dose and Time Response

Dose response curves were constructed to test the echogenicity of the microcapsules prepared by each method. This was performed by either a rapid or standard protocol, depending on the potential of the capsule preparation method to yield highly echogenic capsules suitable for further study. A rapid, cumulative dose response method did not take into account the gradual destruction of the capsules, and thus loss of the signal over time. The custom-made vessel was filled with 50 mL of PBS (25° C.) and adjusted to pH 7.4. The first dose was weighed and added to the PBS in the vessel, and the acoustic enhancement was measured. Every dose there after was added in a cumulative fashion to the same 50 mL of PBS. In the second more time-consuming standard method, capsules were weighed and placed in the custom vessel with 50 mL of PBS (25° C.), and the acoustic enhancement was measured immediately. The PBS with the suspended capsules was discarded, and the procedure was repeated for each increased dose.

Time-response curves were constructed to test the destruction of the capsules and loss of signal over time. The capsules were weighed and placed in 50 mL of PBS (25° C.), pH 7.4, the acoustic enhancement was measured immediately after the capsules were placed in PBS, time was recorded as zero minutes, and acoustic enhancement was measured every 5 min thereafter for 20 min.

All time and dose response curve values are based on an average of three readings each from three different sample preparations (n=9).

The results of the experiments presented in this Example are now described.

### Example 1

### Fabrication of Non-Porous Microcapsules and/or Nanocapsules

The goal of this research is to produce small polymer contrast agents that are echogenic (>8 dB) that can pass unimpeded through mammalian vasculature and pass through fenestrated blood vessels to gain access to tumors and other cancerous growths. The double emulsion method was chosen as the capsule fabrication method for several reasons; it is ideal for encapsulating hydrophobic substances (camphor) in the organic phase and hydrophilic substances (ammonium carbonate) in the aqueous phase, the size of the capsules can be easily modified to the desired size range by small adaptations to the process (using different solvents, surfactants, and homogenization speeds and time) and it produces uniform, spherical capsules. The method can be adapted to encapsulate water-soluble or water-insoluble drugs to address the future goals to use this agent as a drug delivery vehicle.

The two polymers were both 50:50 co-polymers of lactic and glycolic acid with roughly the same molecular weight, 44 kd and 47 kd for RG 504H and DL 3A, respectively. These properties were kept relatively constant because the co-polymer ratio and molecular weight of the polymer are determining factors in polymer degradation and thus microcapsule breakdown. The 50:50 polymer was chosen because it has a relatively short degradation time (weeks to months), which is compatible with the therapeutic need (the agent should be eliminated from the body once it has served its required purposes). Both polymers studied had acid end groups. The acid end groups make the polymer more hydrophilic and increase its ability to uptake water and thus increase degradation time. Although this was not a crucial factor in capsule preparation, its presence in both polymers was important for consistency of the experiment. The inherent viscosity (I.V.) of the RG 504H polymer, 0.49 dL/g, was slightly higher then that of the DL 3A polymer, 0.38 dL/g. The difference between the two polymers was the polydispersity (PDI), which is a measure of the molecular weight distribution of the polymer. The PDI of the RG 504H polymer, 3.22 was greater then the PDI of the DL 3A polymer, 1.8. While the differences in I.V. and/or PDI of the two polymers played a role in microcapsule morphology, they were not the only determining factors. The surfactant (PVA) properties were shown to play an important role.

To evaluate the effect of PVA molecular weight, capsules were prepared with three different PVA molecular weights, (133,000, 25,000, and 6000) and percent hydrolysis (ranging from 80 to 90; Table II). PVA is produced by polymerization of vinyl acetate, which is then hydrolyzed to PVA. Percent hydrolysis refers to the amount of poly vinyl acetate that is hydrolyzed and hence the number of hydroxy groups present on the surfactant. Higher percent hydrolysis of PVA generally yields a stronger surfactant; however, there is a trade-off between percent hydrolysis and molecular weight. The higher the molecular weight the more viscous the solution and thus the stronger the surfactant strength.

Microcapsules RG133, RG25, and RG6 were prepared with the RG 504H polymer, without camphor, and with three different surfactants. RG133 capsules, prepared with the 133,000 MW PVA, produced spherical capsules that were attached to a sponge-like matrix assumed to be PVA. Separation of the capsules was difficult and not pursued. Microcapsules, RG25, were prepared with the 25,000 MW PVA, and formed spherical, uniform capsules with a smooth surface. The microcapsule yield was high (85-90%). Microcapsules were also prepared with 6000 MW PVA, RG6, the results were unexpected with large visible dents throughout the microcapsule preparation. The microcapsule yield was also 85-90%.

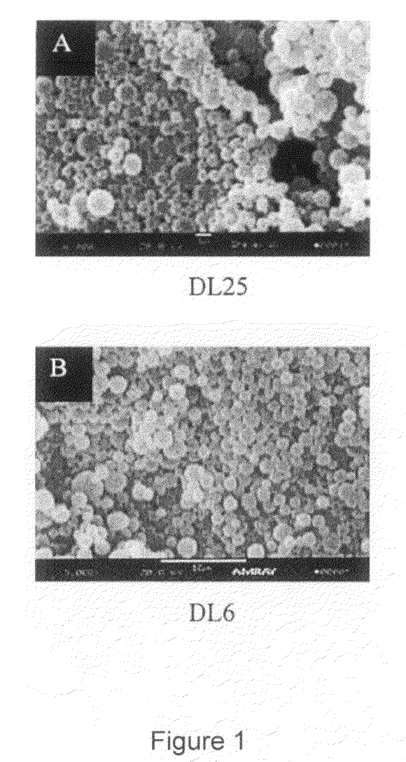

Microcapsules, DL25 and DL6, were prepared with the DL 3A polymer, without camphor and with two different surfactants (FIG. 1). Because of the results obtained with the RG133 sample, the 133,000 MW PVA was not studied further. The microcapsules prepared with the 25,000 MW PVA, DL25 (FIG. 1A), produced uniform, spherical capsules that are directly comparable to the RG25 capsules. The microcapsules prepared with the 6000 MW PVA, DL6 (FIG. 1B), also produced uniform, spherical capsules with a smooth surface; these are very different from the RG6 capsules that had visible dents in the surface morphology. Both preparations gave a capsule yield of 85-90%. Again, if these capsules were sufficiently echogenic they would be ideal ultrasound contrast agents.

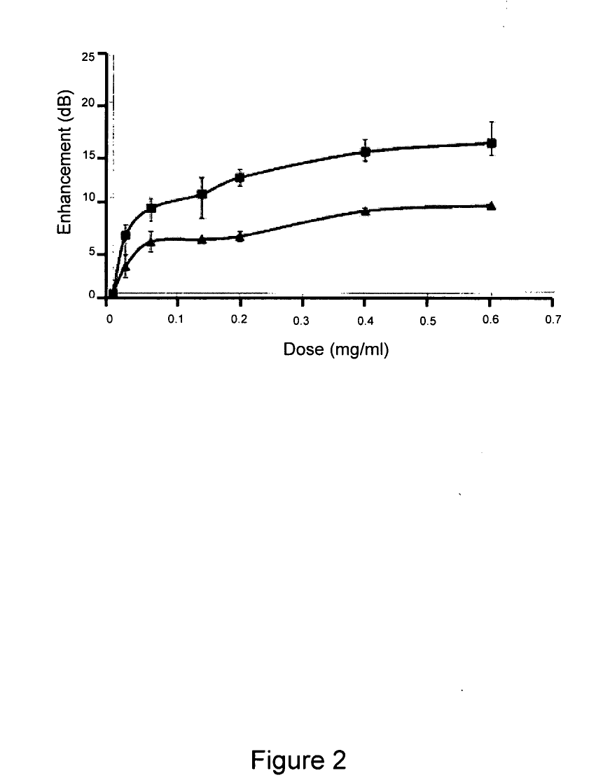

Because of the dented capsules produced with the RG 504H polymer made with the 6000 MW PVA, all further work to develop a contrast agent was pursued with the DL 3A polymer, which consistently produced smooth surface capsules with both the 6000 and 25,000 MW PVA and is also more cost effective than the RG polymer. The DL6 and DL25 capsules, seen in FIG. 1A and FIG. 1B, respectively, were tested for their echogenic properties in vitro. A cumulative dose response curve was constructed to test the echogenicity of the capsules at 5 MHz (FIG. 2). The results show that at the highest dose chosen (0.6 mg/mL) the DL25 capsules gave an average acoustic enhancement of 9.1 dB (+0.14, −0.14) whereas the DL6 capsules gave and enhancement of 15.6 dB (+2.21, −1.27). It is hypothesized from these results (6.5 dB difference between the two samples) that the internal structure of the DL6 and DL25 capsules may be different. The DL6 capsules are expected to have a significantly more porous interior compared with the DL25 capsules. However, the acoustic enhancements produced with the DL25 and DL6 capsules were not high enough to be clinically significant (20-30 dB).

### Example 2

### Fabrication of Porous Microcapsules and/or Nanocapsules

A novel method of developing hollow/porous microcapsules was investigated to increase the acoustic enhancement properties of these capsules. Encapsulating dissolved camphor and later subliming the camphor allows the development of hollow capsules with small diameters (less than 5 μm). Camphor was chosen since it is soluble in the oil phase, and easily removed by sublimation.

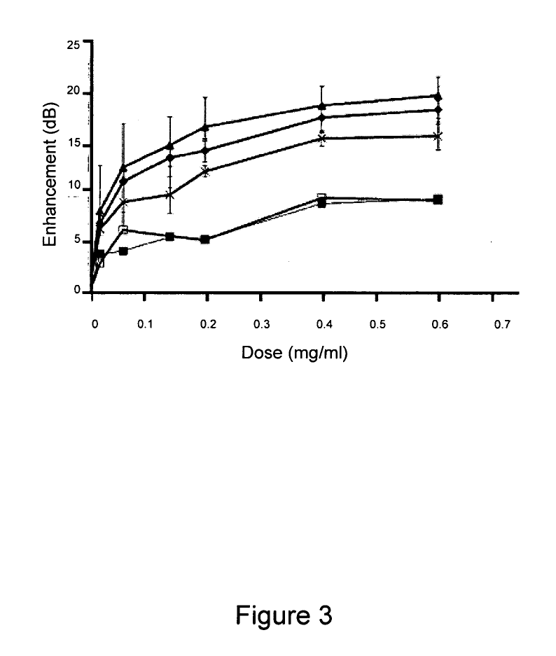

Cumulative dose response curves of the capsules prepared with camphor (0.05, 0.1, 0.25, and 2.5 g) and 25,000 MW PVA (DL25C) were compared with the cumulative dose response curve of the DL25 capsules (without camphor; FIG. 3). Capsules prepared with 2.5 g of camphor had a strong smell of camphor, and it was assumed that lyophilization for 24 hours was not sufficient to remove all the encapsulated camphor. These capsules gave an acoustic enhancement of 9.1 dB at a 0.6 mg/mL dose, similar to that of the capsules prepared without camphor. Capsules prepared with 0.1 g of camphor gave an acoustic enhancement of 15.3 dB at the 0.6 mg/mL dose. These capsules had a slight smell of camphor, indicating that not all the camphor was removed. Capsules prepared with 0.05 g of camphor gave an acoustic enhancement of 19.3 dB with the 0.6 mg/mL dose. These capsules did not smell of camphor, and it was assumed that the high acoustic response was due to the removal of the majority of the encapsulated camphor. Capsules prepared with 0.025 g of camphor did not smell of camphor, and gave an acoustic enhancement of 17.9 dB, with the 0.6 mg/mL dose. Differences in the dose response curves were found to be statistically significant using a 2-way ANOVA (p<0.01), and the best results were obtained when encapsulating 0.05 g of camphor.

Capsules were also prepared with 0.05 g of camphor and the 6000 MW PVA (DL6C). The acoustic enhancement of the capsules was studied, however a cumulative dose response was difficult to construct because the capsules were very unstable, but it was determined that the acoustic properties of the capsules were poor with only about a 12 dB enhancement with the 0.6 mg/mL dose. This enhancement is about 3.5 dB lower then the DL6 capsules. The preparation with camphor and 6000 MW PVA has resulted in weak capsules, capsules that do not have a strong enough shell to remain intact when suspended in PBS and exposed to ultrasound.

The capsules that have shown the greatest enhancement thus far were those prepared with the DL 3A polymer, the 0.05 g of camphor and the 25,000 MW PVA (DL25C). A noncumulative dose (one in which dose of capsules. The contrast agents are relatively stable over time at 25° C. with only minor bubble destruction (4 dB loss) seen in 20 minutes, much longer than would be needed for diagnostic imaging. Although these results are positive, an additional step, encapsulation of ammonium carbonate, was added to further improve the fabrication method of the hollow capsules to allow for reduction of the required dose. Ammonium carbonate was chosen since it is soluble in the aqueous phase and easily removed by freeze drying, when it breaks down to ammonia, carbon dioxide, and water.

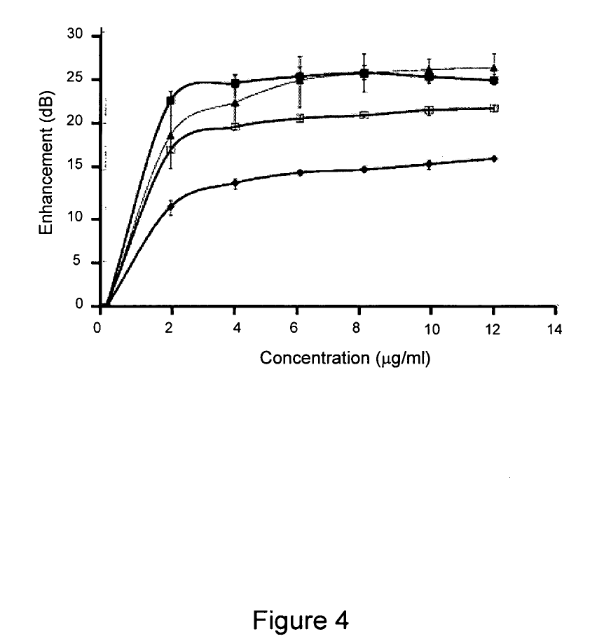

In vitro acoustic studies, through construction of cumulative dose response curves, indicated that the encapsulation of ammonium carbonate in the water phase along with the camphor in the organic phase (DL25AC) does not increase the maximum achievable acoustic enhancement; however it dramatically decreases the total dose needed to reach that value from 0.6 to 0.004 mg/mL. In vitro time response acoustic studies show that the capsule stability is good (loss of only 4 dB over 20 minutes). A cumulative dose response at 2.25, 5, 7.5, and 10 MHz was constructed from capsules prepared with 0.05 g of camphor and 4 w/v % ammonium carbonate solution (FIG. 4). Acoustic enhancements of 15.9 (+0.30, −0.22), 24.5 (+0.67, −0.49), 25.9 (+1.52, −1.76), and 21.4 (+0.30, −0.39) dB are seen at 2.25, 5. 7.5, and 10 MHz, respectively, with a dose of 8 μg/mL. These results gave good acoustic enhancement for a low dose. The size distribution showed that 99.6% of capsules were between 0.4 to 4.0 μm, with a mean diameter of 1.210 μm. The size distribution and uniformity of these microcapsules along with their echogenic nature make them ideal contrast agents for intravenous use. SEM of the surface morphology shows smooth spherical capsules. Acoustic disruption of the spheres with a probe sonicator shows their hollow interior and the reason for their echogenicity.

During the course of the experiments, some initial inconsistent results led to the hypothesis that the temperature of the PVA used during the capsule fabrication process had an effect on the echogenicity of the agent. Thus capsules were prepared at three different PVA temperatures, 4, 26, and 45° C. The dose response of the capsules was tested and compared and indeed results were found to be statistically significant using a 2-way ANOVA (p<0.01), with the best results obtained with the PVA solution set at 4° C.

Using a dose of 0.03 mg/mL, the capsules prepared with PVA at 45° C. had the lowest acoustic enhancement of 0.03 mg/mL (8.599 dB, +3.32, −3.07) in comparison with those prepared with PVA set at room temperature, 26° C. (16.23 dB, +3.53, −3.07) and at 4° C. (19.55 dB, +0.278, −0.491). It was interesting to note that the +error between samples was much smaller with the cold PVA than with either the warm or room temp PVA. To our knowledge, the criticality of surfactant temperature has not been reported previously.

It has been shown that fabrication of porous microcapsules can be developed with small adaptations to the double emulsion process. The PVA temperature study has stressed the need and significance for use of cold PVA during the fabrication process. The capsules produced have shown positive acoustic in vitro results. Their size distribution should allow for unimpeded passage through the circulatory system.

### Example 3

### Fabricating a Microcapsule and/or Nanocapsule Comprising a Therapeutic Agent

A microcapsule and/or nanocapsule may be fabricated that comprises a therapeutic agent by any method known in the art. Examples include wet or dry drug adsorption, encapsulation or incorporation, conjugation or attachment.

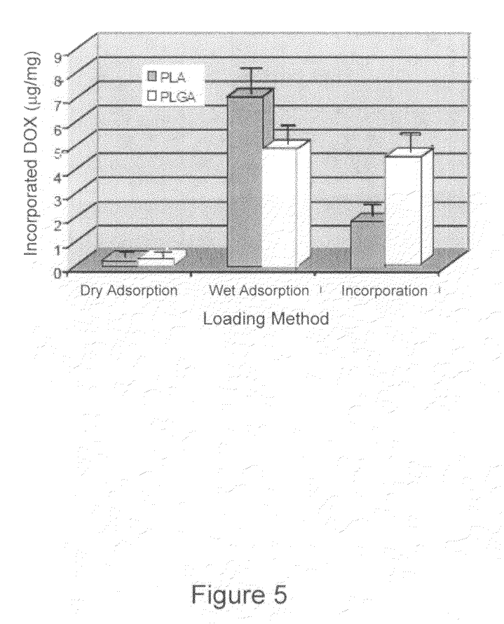

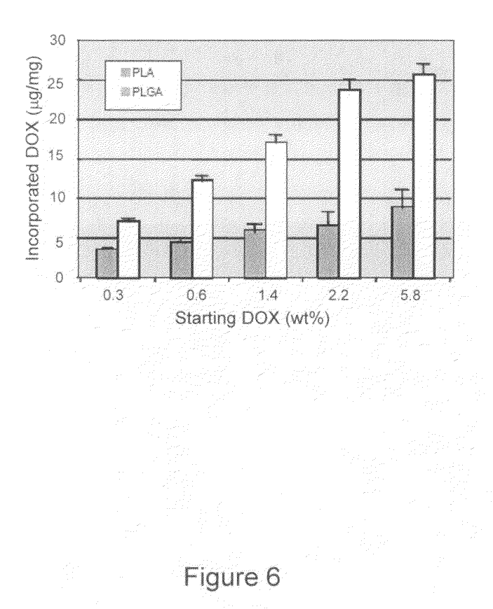

Drug encapsulation was found to be influenced by both polymer type (PLGA retained more drug than PLA), and initial starting concentration of drug (% of starting dose increased as drug concentration increased). Representative graphs are shown in FIG. 5 and FIG. 6. The echogenicity of the drug-loaded capsules starting at 20 dB for unloaded capsules is in the order unloaded˜incorporated>wet adsorption>dry adsorption.

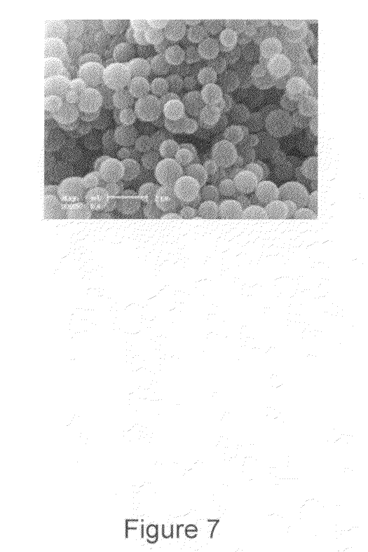

Data indicate that a nanocapsule and/or microcapsule can be loaded with drug, without altering the excellent echogenic properties. FIG. 7 shows the drug-loaded capsules under a scanning electron microscope. Note the small (average diameter 1.2 μm) uniform size, ideal for free passage through the smallest capillaries.

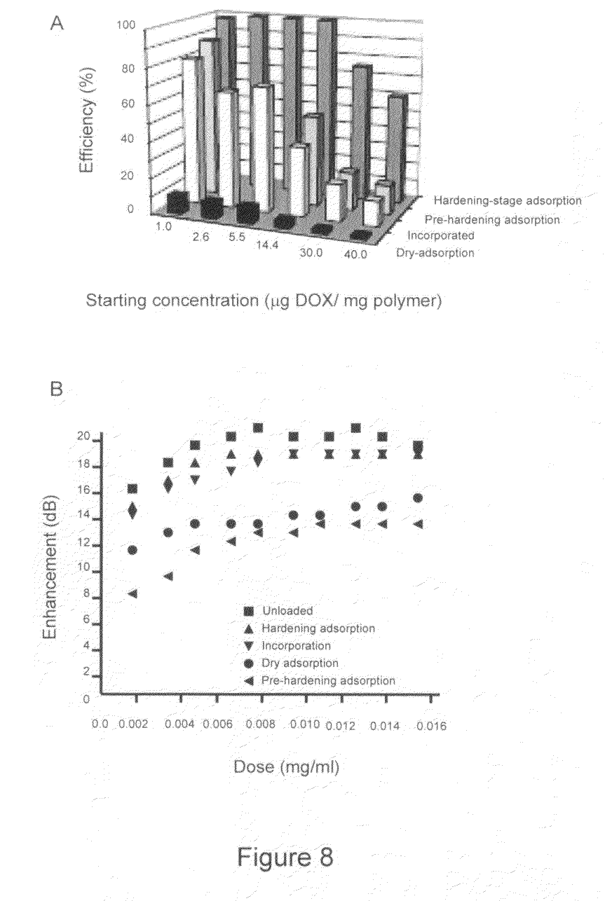

A new method of drug loading was investigated which involved adding drug to the nascent capsules prior to the hardening stage. High loadings were achieves, comparable to thoseobtained by the incorporation method (FIG. 8A shows curves for the polymer PLA), but the resulting capsules had diminished echogenicity (FIG. 8B), similar to dry adsorption.

A. Peptide Attachment to a Microcapsule and/or Nanocapsule

The peptide conjugation reaction is a modification of carbodiimide chemistry. 1-Ethyl-3-13-dimethylamino-propyl carbomiidie (EDC; 0.005 grams) and 0.0027 grams of N-hydroxysuccinimide (NHS) were dissolved in 10 ml of 2-[N-morpholino]ethanesulfonic acid (MES) buffer (pH 6.5). Microcapsules and/or nanocapsules (1 gram) were suspended in the mixture and shaken on a shaker for 15 minutes to activate the surface of the polymer and prepare the polymer for peptide attachment. Peptide (150 μg) was added and the mixture was shaken for an additional 3 hours. Following shaking, the mixture was centrifuged to collect the capsules. The capsules were then washed with deionized H₂O and centrifuged again to collect capsules after washing. Collected capsules were frozen at −85° C. for 30 minutes and then lyophilized to dry the capsules and to remove any additional reagents.

B. Chemotherapeutic Agents

Doxorubicin is one of the more effective chemotherapeutic agents for liver cancer and is also naturally fluorescent (488 nm excitation, 550 nm emittance). Loading the therapeutic agent payload by incorporation during the w/o/w production stage was performed.

The prodrug Irinotecan is the hydrochloride salt of a semisynthetic derivative of camptothecin, a cytotoxic, quinoline-based alkaloid extracted from the Asian tree Camptotheca acuminata. It is converted to its biologically active metabolite 7-ethyl-10-hydroxy-camptothecin (SN-38) by a carboxylesterase-converting enzyme. SN-38 is a topoisomerase I inhibitor, eventually triggering apoptotic cell death. It is classified as an S-phase-specific agent.

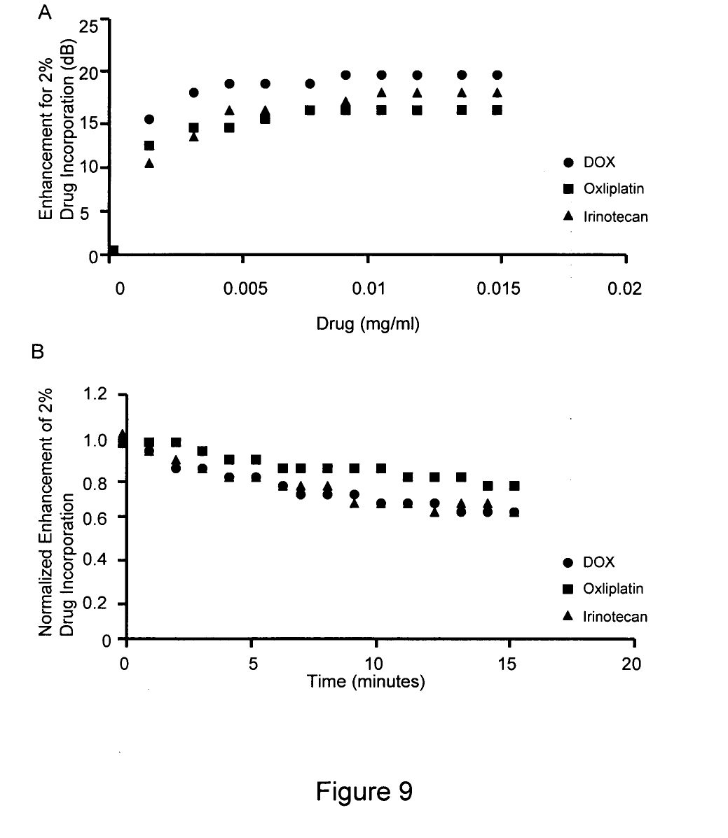

Oxaliplatin is an anti-neoplastic that acts as an alkylating agent. It is often given in combination with the vitamin leucovorin. It is usually followed by an injection of 5-fluorouracil. Both drugs were loaded by incorporation of a 2 wt % drug load onto PLA contrast agents and the echogenicity and stability of the resulting drug-loaded agents were measured and compared with the data for doxorubicin. No statistical difference in enhancement (FIG. 9A) or stability (FIG. 9B) was found in the three drugs combinations.

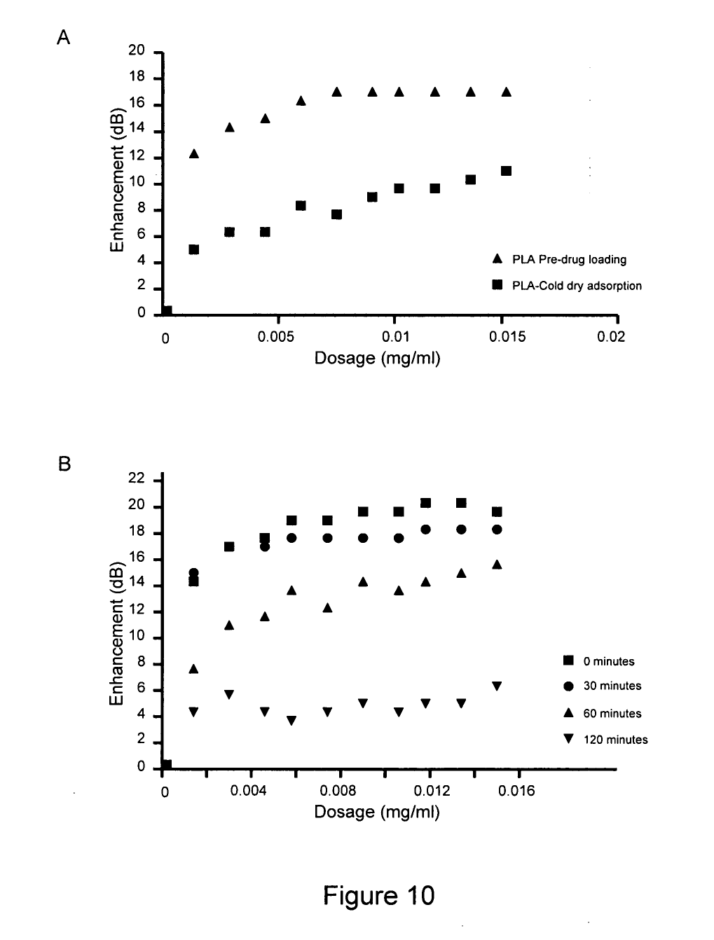

Loading of drug by adsorption yielded the lowest encapsulation efficiency, but increased loading times lead to substantial loss of echogenicity. The activity loss may be due to polymer hydrolysis during the aqueous loading step, and that loading at lower temperature would slow the hydrolysis reaction to a far greater extent than lowering the adsorption. Reaction rate halves for every 10° C. temperature drop. Dry contrast agent sample was soaked in DOX Solution for 24 hours at 4° C. Samples were then washed 3× with deionized water, collected and freeze dried. The samples loaded at 4° C. preserved far greater echogenicity (FIG. 10).

One of the oldest and well-established chemotherapeutics is 5 fluorouracil. It is a water soluble analogue of the naturally occurring base, uracil. The half life is very short, of less than 5 min. in the body, and thus would be an ideal candidate for encapsulation. It is often given with folinic acid, which boosts its activity, and in combination with Oxaliplatin) named FOLFOX. 5FU loaded well onto the capsules, the maximum amount was by wet adsorption as with Doxorubicin. As the amount of drug loading increased the echogenicity decreased (results not shown), with a drop for example from a maximum enhancement of 20 dB to 15 dB for 4% incorporation. Stability over time, under constant insonation was not severely affected.

### Experimental Example 4

### In Vivo Administration of Nanocapsules and/or Microcapsules

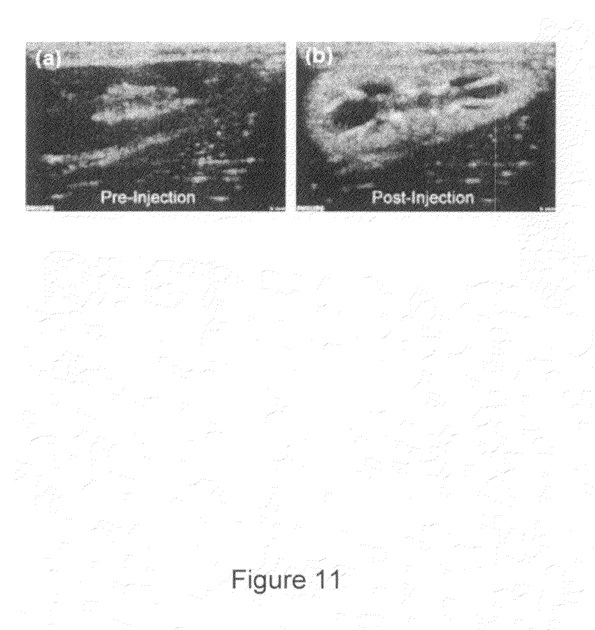

FIG. 11 is an ultrasound image of a rabbit kidney before, (a) and after, (b) administration of a dose of 0.1 mL/kg (0.04 g of CA/mL), loaded with 2% (wt/wt) of sudan black, a model for the highly hydrophobic chemotherapeutic Paclitaxel. The agent highlights the vasculature of the cortex, revealing the structure.

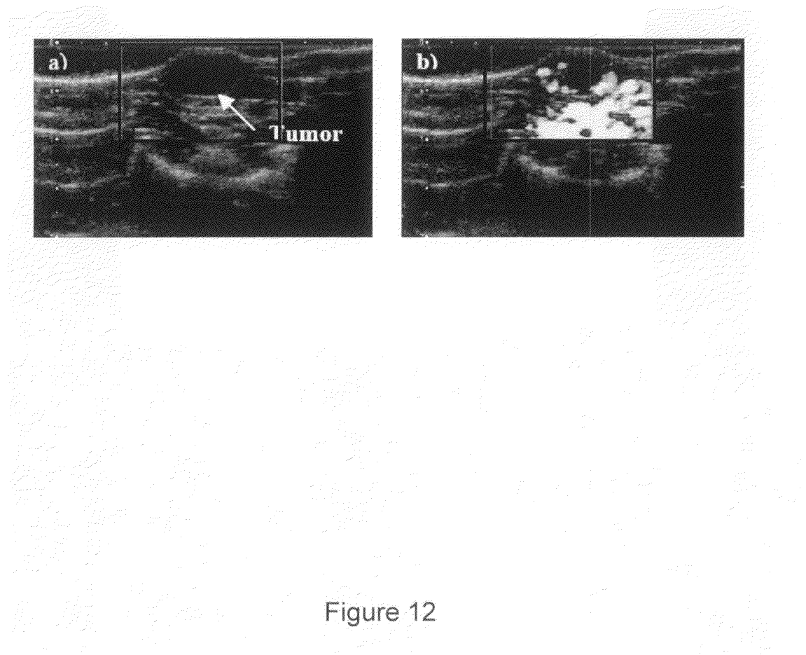

Doxorubicin-loaded nanocapsules and/or microcapsules maintain excellent echogenicity in vivo, comparable to unloaded capsules. FIG. 12 depicts a micro flow Doppler image (boxed portion) of a subcutaneous melanoma tumor implanted into nude mouse before (FIG. 12A) and after (FIG. 12B) after injection of Doxorubicin-loaded nanocapsules and/or microcapsules. Doxorubicin-loaded nanocapsules and/or microcapsules can be seen penetrating the tumor. A large number of Doxorubicin-loaded nanocapsules and/or microcapsules can be seen in the vessels feeding the tumor (lower right portion of boxed area of FIG. 12B).

### Experimental Example 4

### Ultrasound Triggered Drug Release from a Nanocapsule and/or Microcapsule

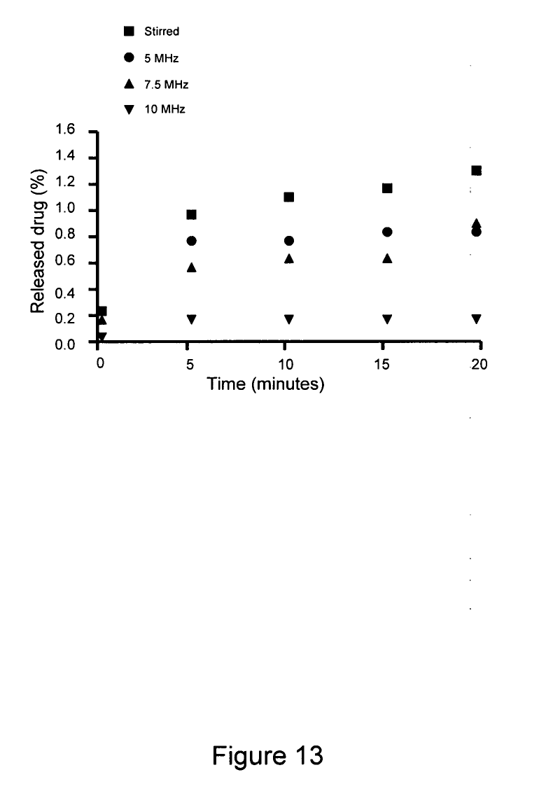

When drug is loaded into the shell of a nanocapsule and/or microcapsule, it is demonstrated herein that it can be released into simulated physiological fluid (PBS) (FIG. 13). The graph demonstrates that when insonated with ultrasound of various frequencies, all of which fall within the medical imaging range, (5 MHz, 7.5 MHz and 10 MHz), the drug is released to various different degrees, and to a significantly greater extent than in the absence of ultrasound (stirred only). The release experiments are conducted in the same in vitro apparatus used for acoustic characterization of the nanocapsules and or microcapsules. It consists of a one-dimensional pulsed A-mode US setup with a single element, broadband, 12.7 mm element diameter, 50.8 mm spherically focused transducers with center frequencies of 2.25, 5, 7.5, and 10 MHz. The transducers are inserted in a deionized water bath (37° C.) and focused through an acoustic window of a 100 ml custom-made sample vessel. A pulser/receiver is used to pulse the transducers at a pulse repetition frequency of 100 Hz. The received signals are amplified to 40 dB and fed to the digital oscilloscope. The digitized data is stored and analyzed using Labview.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of selectively contacting a liver cancer cell with a therapeutic agent in an individual with liver cancer, said method comprising administering a composition comprising a polymer-based, echogenic nanocapsule and/or microcapsule to said individual, wherein said therapeutic agent is attached to, adsorbed to, encapsulated in, or any combination thereof, said nanocapsule and/or microcapsule, wherein said nanocapsule and/or microcapsule further comprises a targeting moiety, wherein said targeting moiety interacts with a target molecule present on a liver cancer cell, thereby contacting said liver cancer cell with said therapeutic agent.

2. The method of claim 1, wherein said therapeutic agent is released in the liver of said individual by degradation of said polymer-based nanocapsule and/or microcapsule.

3. The method of claims 1, wherein said individual is human.

4. The method of claim 2, wherein said degradation is enhanced by ultrasound.

5. The method of claim 1, wherein the distribution of said nanocapsule and/or microcapsule comprising a therapeutic agent is monitored using ultrasound, wherein when said nanocapsule and/or microcapsule is detected in the liver, the selective release of said therapeutic agent in the liver is triggered by the rupture of said nanocapsule and/or microcapsule with ultrasound.

6. A method of treating liver cancer, said method comprising the steps of:
a) administering to an individual with liver cancer a composition comprising a nanocapsule and/or microcapsule, wherein said nanocapsule and/or microcapsule comprises a therapeutic agent, wherein said therapeutic agent either directly or indirectly inhibits the viability, growth, or proliferation of said cancer; and
b) monitoring the in vivo distribution of said nanocapsule and/or microcapsule using ultrasound to detect said nanocapsule and/or microcapsule in the liver; wherein when said nanocapsule and/or microcapsule is detected in the liver, said therapeutic agent is selectively released in the liver by enhancing the biodegradation of said nanocapsule and/or microcapsule by insonation of the liver.

7. The method of claim 6, wherein said individual is human.

8. A method of treating liver cancer, said method comprising the steps of:
a) administering to an individual with liver cancer a composition comprising a nanocapsule and/or microcapsule, wherein said nanocapsule and/or microcapsule comprises a therapeutic agent, wherein said therapeutic agent either directly or indirectly inhibits the viability, growth, or proliferation of said cancer, and wherein said nanocapsule and/or microcapsule further comprises a targeting moiety, wherein said targeting moiety interacts with a target molecule present on a liver cancer cell; and
b) monitoring the in vivo distribution of said nanocapsule and/or microcapsule using ultrasound to detect said nanocapsule and/or microcapsule is in the liver, wherein when said nanocapsule and/or microcapsule is detected in liver, said therapeutic agent is selectively released in the liver by enhancing the biodegradation of said nanocapsule and/or microcapsule by insonation of the liver.

9. The method of claim 8, wherein said individual is human.

10. A method of treating liver cancer, said method comprising the steps of:
a) administering to an individual with liver cancer a composition comprising at least one population of a nanocapsule and/or microcapsule; wherein said population essentially comprises a single therapeutic agent, a single targeting moiety, or a combination thereof, wherein said therapeutic agent either directly or indirectly inhibits the viability, growth, or proliferation of said cancer, and wherein said targeting moiety interacts with a target molecule present on a liver cancer cell; and
b) monitoring the in vivo distribution of said population of a nanocapsule and/or microcapsule using ultrasound to detect said population is in the liver, wherein when said population is detected in liver, said therapeutic agent is selectively released in the liver by enhancing the biodegradation of said nanocapsule and/or microcapsule by insonation of the liver.

11. The method of claim 10, wherein said individual is a human.
